(19) United States
(12) Patent Application Publication
(10) Pub. No.: US 2024/0148946 A1
(43) Pub. Date: May 9, 2024

(54) MIXING APPARATUS FOR PRODUCING SOLUTIONS, IN PARTICULAR OF DIALYSIS OR INFUSION TREATMENTS

(71) Applicants: Marvin LOHSE, Bielefeld (DE); Matthias WESSELER, Melle (DE)

(72) Inventors: Marvin LOHSE, Bielefeld (DE); Matthias WESSELER, Melle (DE)

(73) Assignee: Marvin LOHSE, Bielefeld (DE)

(21) Appl. No.: 18/281,452

(22) PCT Filed: Mar. 14, 2022
(86) PCT No.: PCT/EP2022/056575

§ 371 Date: Sep. 11, 2023

(30) Foreign Application Priority Data
Mar. 13, 2021 (DE) 10 2021 001 350.6

(51) Int. Cl. A61M 1/16

(52) U.S. Cl. CPC A61M 1/1656

(57) ABSTRACT

Mixing apparatus for producing solutions for medical treatment, in particular for administering dialysis or infusion solutions.

The present invention relates to an apparatus and a method for preparing and administering solutions, in particular dialysis and infusion solutions, with which the provision of individualized compositions is possible. The apparatus is preferably intended to ensure that the concentration of individual substances is not inadvertently fallen below or exceeded.

There are various methods for the blood treatment of a patient, in particular for the elimination of substances from the blood of a patient and for removing water from the blood of a patient. These methods are referred to as dialysis methods. A further method to compensate for insufficient blood volume is the infusion of a solution into the bloodstream of the patient.

In hemodialysis, excess substances and water are eliminated from the patient's blood in an extracorporeal blood circuit. The extracorporeal blood circuit typically contains a dialysis filter, which comprises two chambers separated from one another by a semipermeable membrane. The patient's blood is passed through one of the chambers, and a flushing liquid (dialysate) is passed through the other chamber. Because of a substance concentration gradient between the patient's blood and the flushing liquid, substances and water are transported through the semipermeable membrane by the physical effects of diffusion and osmosis.

In a hemofiltration, the patient's blood is passed through an extracorporeal blood circuit in a similar way to hemodialysis. In the dialysis filter, the substance transport through the semipermeable membrane is caused by a pressure gradient, that is to say convectively. The liquid removed by the pressure gradient needs to be replaced either before or after the dialysis filter by the infusion of replacement solution into the blood circuit.

The combination of hemodialysis and hemofiltration methods is known as hemodiafiltration.

The flushing liquid and the replacement solution may be connected to a blood treatment unit either as a usable solution or as a concentrate. If a concentrate is used, it needs to be diluted in the blood treatment unit in a defined ratio with water to form a usable solution.

In order to prevent the precipitation of substances with low solubility during delivery, the flushing liquid or the replacement solution are usually delivered while being separated into two or more different constituents, and only mixed to form the usable solution in the blood treatment unit. These constituents will be referred to below by way of example as constituent A and constituent B, although more constituents may also be used depending on the provider of the dialysis treatments. Flushing liquid may be processed by the blood treatment unit using sterile filtration to form a replacement solution.

Modern blood treatment units make use of the mixing and dilution of constituents A and B by being able to generate a concentration profile over the treatment time using a lower or higher dilution of constituents A and B. If constituent A is diluted less, for example, there will be a higher concentration of all substances contained in A in the flushing liquid. The relative proportion of the substance components of A with respect to one another does not vary, however, and only the relative proportion of the substance components of A to the substance components of B varies.

In a dialysis treatment according to the prior art, one of the constituents consists of about 95% NaCl and the other constituent consists entirely of sodium bicarbonate. By altering the dilution ratios, it is therefore possible to vary the concentration of sodium chloride or sodium bicarbonate in the final flushing liquid, while the composition of the additional 5% of substances present in the NaCl solution experiences a very low quantitative change in absolute terms.

Since the flushing liquid can be processed to form a replacement solution using sterile filtration by means of sterile filters, this principle also applies equally for the replacement solution. Entirely mutually independent variation of all the substances in the flushing liquid or in the replacement solution is not used according to the prior art.

It is desirable to find a further improved way of adapting the substance composition of the flushing solution or replacement solution. This is in particular so as to offer a patient an individualized treatment, for example an adaptation of the solution to their individual physiology and individual clinical picture. Furthermore, the patient's balance of substances in the intracellular and extracellular spaces changes during a dialysis treatment. This is not taken into account satisfactorily in the dialysis therapy according to the prior art.

According to the prior art, therapy adaptation is carried out by having to provide various concentrated and usable solutions having different formulations and store them in the dialysis stations. For each patient, a suitable formulation then needs to be selected in advance (there are >50 different formulations with different dilution ratios on the market), and these then need to be connected to the blood treatment unit in the form of the concentrate or in usable form. This leads to great logistical outlay since a different formulation needs to be connected to the blood treatment unit before and after each dialysis treatment.

Logistically, it would be more favorable to supply the blood treatment units by using a central supply in which a very large reservoir is connected via liquid lines to the blood treatment units. In this case, however, the patients are all treated with the same composition of the flushing liquid or replacement solution. Individualization would therefore not be possible.

Apparatuses are already known which are used to produce the flushing liquid or replacement solution at the blood treatment unit, for example from DE102013102914 A1. In this apparatus, the various constituents (usually salts or solutions thereof) are conveyed from mutually separate storage containers directly into the pure water line and the mixing of the flushing liquid is thereby generated. If the salts are in a pure form in the containers, a number nᵢ=nₛ of fluid inlets is correspondingly needed for a number nₛ of substances to be varied independently. This route, however, is to be regarded as critical in terms of patient safety since legally approved or therapeutically necessary limits could be exceeded or fallen below in the event of a malfunction of the mixing apparatus. If there is highly concentrated potassium and chloride in one of the containers, for example, the patient could be treated with an excessive dose thereof, which in the worst case would cause death of the patient.

The object of the invention is therefore to overcome or at least mitigate at least one of the disadvantages of the prior art.

In particular, an advantage of the apparatus according to the invention is that it allows an improved blood treatment by the ability to vary the substance composition of some or all solutions intended for the treatment of the patient, freely and mutually independently before and/or during the treatment, without the possibility that the specified limits in the substance concentration of individual substances in the solution usable for the patients may be exceeded or fallen below.

For this purpose, it is advantageous that the apparatus according to the invention is connected upstream of the dosing apparatus in the blood treatment unit, to which the acidic component (component A) is connected in the case of a blood treatment unit according to the prior art. In this case, depending on the embodiment, the mixing apparatus may either be integrated into the blood treatment unit (cf. FIG. 2) or located outside the blood treatment unit as an additional module. In the latter case, this additional module is preferably connected to the blood treatment unit via the fluid outlet.

The technical apparatus has at least two fluid inlets for base formulations, each inlet having at least one dosing apparatus (for example pumps, valves). By this dosing apparatus, the fluid inlets are in fluidic connection with a common liquid line system. This liquid line system may, in one preferred embodiment, be configured as a reservoir or comprise a reservoir. A regulating or control device may monitor the physical state of the apparatus (for example flow rate, pressure, temperature, conductance) by means of a sensor. The dosing apparatus may therefore be controlled so that the solution thus produced, for example for dialysis treatments or infusion treatments, corresponds to the criteria established by the blood treatment unit and the data input interface.

In one preferred embodiment, the mass or volume flow rate is specified by the blood treatment unit, while the composition of the mixed solution is specified by input of patient data into the data input interface. The control or regulating device will thus be able to produce the solutions for the patient treatment in a given amount and given composition. The usable solution may then be taken from the apparatus through a fluid outlet.

In one preferred embodiment, the fluid outlet debouches into the dosing apparatus of the blood treatment unit, with which the flow rate of the acidic component (component A) is controlled in the case of blood treatment units according to the prior art.

In one preferred embodiment, the number of fluid inlets (A1, A2, A3, A4, An) is contingent on the number of substances which are intended to be varied mutually independently in the finally mixed solution at the fluid outlet, and is described by the following formula:

nᵢ=2ⁿ^{ₛ}
nᵢ number of fluid inlets
nₛ number of substances which are intended to be varied

For example, if the intention is to vary two substances S_1 and S_2 of the solution for the dialysis or infusion treatment within the limits min and max, four solutions (also referred to as “base formulations” in what follows) need to be connected to four fluid inlets (A1, A2, A3, A4), in which the two substances to be varied may be present in the following concentration:

| Inlet A₁ | Inlet A₂ | Inlet A₃ | Inlet A₄ |
|---|---|---|---|
| S_{1, min} | S_{1, min} | S_{1, min} | S_{1, min} |
| S_{2, min} | S_{2, min} | S_{2, min} | S_{2, min} |
| S₃ | S₃ | S₃ | S₃ |
| S₄ | S₄ | S₄ | S₄ |
| ↓ . . . ↓ | ↓ . . . ↓ | ↓ . . . ↓ | ↓ . . . ↓ |
| Sₙ | Sₙ | Sₙ | Sₙ |

In this example, usable solutions which differ from one another only in the substance concentrations to be varied are connected to each fluid inlet (A1, A2, A3, A4, An) of the mixing apparatus.

In one preferred embodiment, each of the connected base solutions is a solution approved according to pharmaceutical legislation.

By the control and regulating device, the dosing apparatuses at the fluid inlets are controlled in such a way that, by means of the mixing ratio of the base formulations with respect to one another, the substance composition specified through the data input interface is present within the limits min and max at the fluid outlet and the required volume or mass flow rate is adjusted.

If each individual base formulation per se is approved according to pharmaceutical legislation, even in the event of a complete malfunction of the apparatus there can never be a substance concentration outside the interval min and max at the fluid outlet.

If the limits min and max lie within limits approved for a dialysis or infusion treatment or are selected by the operator of the technical apparatus according to the individual patient risk, although in the event of a malfunction of the technical apparatus the patient will be treated less well, the substance concentrations in the mixed solution at the fluid outlet can never reach a range which endangers the patient.

This is in contrast to the technical apparatus disclosed by DE102013102914A1, in which highly concentrated substances outside the existing approval limits are conveyed directly into a pure water line, so that excessively high or low concentrations are also possible.

All other substances S_{3 . . . n} in the connected base formulations are preferably present at each fluid inlet (A1, A2, A3, A4) in the same substance quantity, and therefore do not change their substance concentration in the mixed solution at the fluid outlet of the technical apparatus.

With reference to the example of a dialysis treatment, potassium and calcium ions may be selected as substances to be varied, while sodium, magnesium and acetate ions and glucose do not vary in this approach. Depending on the therapy, however, any substance may be intended for variation. More than two substances, and likewise only one substance, may also be varied according to the aforementioned formula.

In the case of cationic substances, it should be mentioned that the anionic substances (usually chloride ions) are jointly varied according to electrical neutrality, but are not taken into account in the formulae for determining the number of fluid inlets.

For example, if the intention is to vary the concentration of potassium ions in the finally mixed solution, the chloride ions are jointly varied to the same extent since potassium in solutions for dialysis is usually in the form of dissolved potassium chloride. Nevertheless, even though with potassium and chloride two substances are varied, nₛ=1 is selected since potassium and chloride cannot be varied independently of one another.

In another embodiment, the liquid line system may be configured as a tube system which is intended for single use. This embodiment may be preferred especially for use in small production numbers, for example for a clinical trial of the equipment, since such a tube system as a disposable article can be sterilized well and hygiene can therefore be ensured. The dosing apparatuses may in such a case be configured as peristaltic tube pumps.

### DESCRIPTION OF THE FIGURES

FIG. 1 schematic and exemplary representation of hemodiafiltration according to the prior art

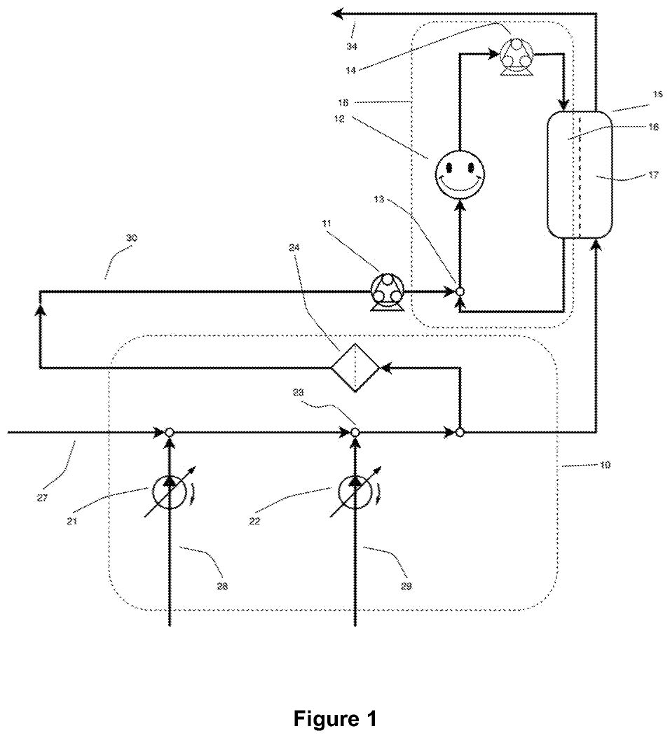

FIG. 2 schematic and exemplary representation of hemodiafiltration using an embodiment of the invention according to the invention, the apparatus being configured so that it is integrated into the blood treatment unit

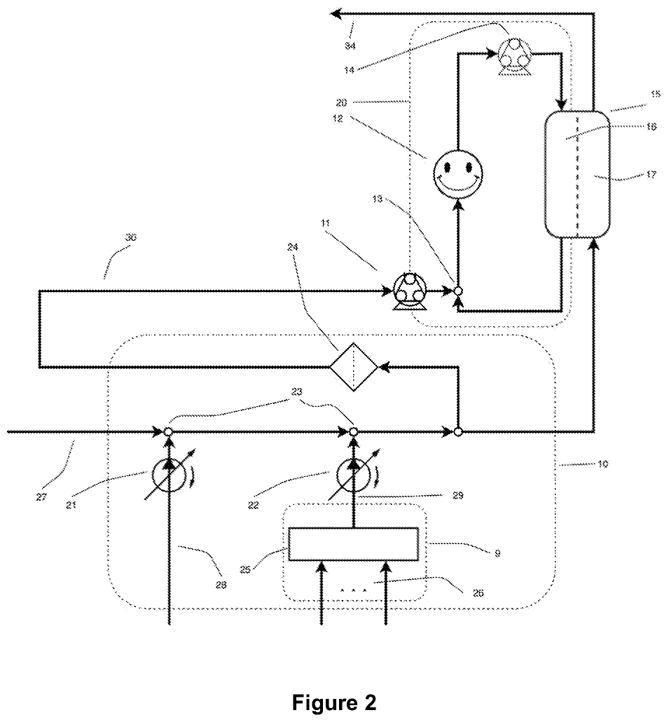

FIG. 3 exemplary embodiment of the mixing apparatus according to the invention

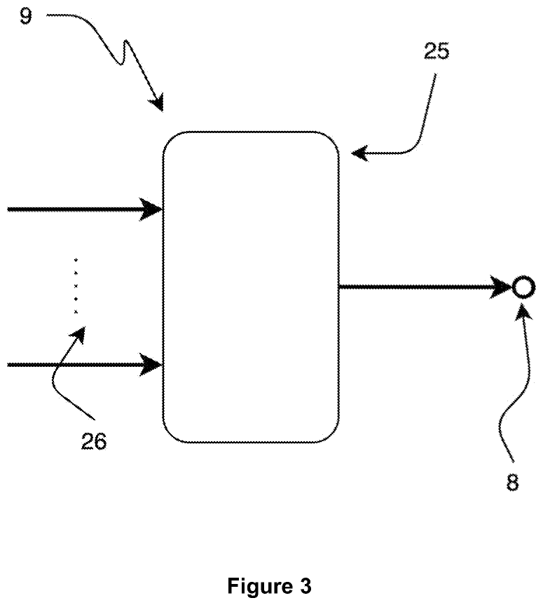

FIG. 4 advantageous embodiment of the apparatus according to the invention, there being four fluid inlets for the independent variation of two substances and the common liquid line system comprising a reservoir with a sensor device

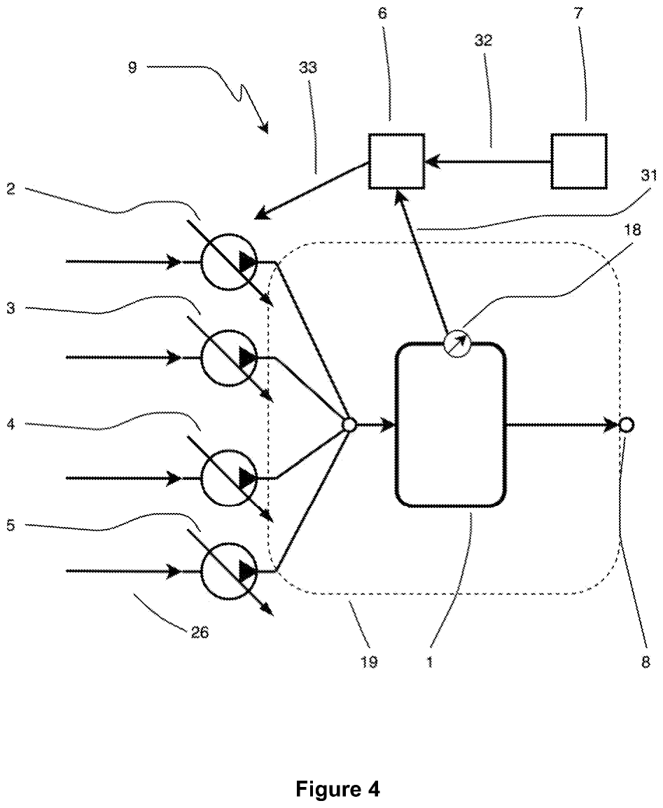

### PREFERRED EMBODIMENTS

1. Technical mixing apparatus for the patient-individual production of usable or concentrated solutions for dialysis or infusion treatments, wherein particular concentration limits of particular substances in the composition of the finally mixed solution cannot be fallen below or exceeded because of the construction of the apparatus, while the substance concentration of the finally mixed solution can be varied before and during the treatment, having:
   at least two fluid inlets (26), to which only base solutions are connected, the latter in turn lying within the concentration limits and differing from one another only in the substance quantities of the substances to be varied at the fluid outlet (8);
   a mixing device (25), which quantitatively mixes the base solutions connected to the fluid inlets in such a way that the mixed solution at the fluid outlet (8) has the required volume or mass flow rate and corresponds in its substance composition to the required substance composition;
   at least one fluid outlet (8), at which the substance composition of the finally mixed solution lies within the composition intervals defined by the base solutions connected to the fluid inlets (26), these intervals, since only base solutions whose substance compositions in turn lie within a particular concentration limits, also only being capable of lying within these concentration limits.
2. Technical mixing apparatus according to embodiment 1, wherein the device is formed from the following constituents:
   a number

nᵢ=2ⁿ^{ₛ}
of fluid inlets (26) for the for the mutually independent variation of a number nₛ of substances contained in the finally mixed solution for dialysis or infusion treatments, to which only base solutions are connected, the latter in turn lying within legal (for example standard approval) or therapeutic limits and differing from one another only in the substance amounts of the substances to be varied at the fluid outlet (8);
   per fluid inlet (26), respectively at least one dosing apparatus (2,3,4,5), the latter being in fluidic connection with a common liquid line system (19);
   at least one common liquid line system (19), which may comprise a liquid reservoir (1) and may contain at least one convectioning device;
   at least one data input interface (7), which can receive data from the user, patient data or data from the blood treatment unit (10) and make these data available to the apparatus (9), and in particular to a control or regulating device (6);
   at least one control or regulating device (6) which controls or regulates the dosing apparatuses in such a way that each base solution connected to the fluid inlet (26) is quantitatively admixed with the common liquid line system (19) so that the solution for dialysis or infusion treatments as the volume or mass flow rate required at the fluid outlet (8) and corresponds in its substance composition to the substance composition defined by the data input interface (7);
   at least one fluid outlet (8) for the finally mixed solution for the dialysis or infusion treatment, which may likewise comprise a dosing apparatus.
3. Technical mixing apparatus (9) according to embodiment 2, wherein the technical apparatus (9) comprises at least one sensor device (18) which measures at least one physical quantity in the mixing apparatus (9) according to the invention and makes this measurement result available to the control or regulating device (6).
4. Technical mixing apparatus (9) according to one of embodiments 1-3, wherein the technical apparatus (9) is configured as an integral constituent of a blood treatment unit (10).
5. Technical mixing apparatus (9) according to one of embodiments 1-3, wherein the technical apparatus is located outside a blood treatment unit and is connected thereto.
6. Technical mixing apparatus (9) according to one of the preceding embodiments, wherein the common liquid line system (19) contains a reservoir (1).
7. Technical mixing apparatus (9) according to one of embodiments 1-6, wherein the number of inputs is set to 4 inputs for mutually independent variation of the concentration of 2 substances at the fluid outlet (8) of the technical mixing apparatus (9).
8. Technical mixing apparatus (9) according to one of embodiments 1-6, wherein the number of inputs is set to 2 inputs for variation of the concentration of one substance at the fluid outlet (8) of the technical mixing apparatus (9).
9. Technical mixing apparatus (9) according to one of the preceding embodiments, wherein the dosing apparatus (26) is configured as a pump or valve.
10. Technical mixing apparatus (9) according to one of the preceding embodiments, wherein the control or regulating unit (6) is configured as a programmable processor.

### LIST OF REFERENCE SIGNS

1 reservoir in the mixing apparatus according to the invention
2 dosing apparatus at inlet A₁
3 dosing apparatus at inlet A₂
4 dosing apparatus at inlet A₃
dosing apparatus at inlet A₄
6 control or regulating unit
7 data input interface
8 fluid outlet
9 technical mixing apparatus according to the invention
10 the blood treatment unit, or its system limit
11 peristaltic replacement solution
12 patient
13 infusion point in extracorporeal blood circuit (postdilution in all figures)
14 peristaltic blood pump
15 dialysis filter
16 blood chamber of the dialysis filter
17 flushing solution chamber of the dialysis filter
18 sensor device
19 liquid line system in the technical mixing apparatus
20 extracorporeal blood circuit
21 flow control device for the alkaline component (B)
22 flow control device for the acidic component (A)
23 Solution points, at which the acidic or alkaline component is respectively mixed with pure water and the previously supplied component
24 sterile filter
25 mixing device
26 inlets Aₙ
27 pure water
28 inlet B
29 inlet A
30 Replacement solution
31 sensor device line
32 composition information line
33 control signal line
34 spent dialysis solution

1. A mixing apparatus for producing solutions to be administered to humans, in particular for dialysis or infusion treatments, having:
at least two fluid inlets (26), to which base solutions are connected, the latter lying within predefined concentration limits and differing from one another only in the substance quantities of the substances to be varied at the fluid outlet (8);
a mixing device (25), which quantitatively mixes the base solutions connected to the fluid inlets in such a way that the mixed solution at the fluid outlet (8) has the required volume or mass flow rate and corresponds in its substance composition to the required substance composition;
at least one fluid outlet (8), at which the substance composition of the finally mixed solution lies within the composition intervals defined by the base solutions connected to the fluid inlets (26).

2. The mixing apparatus as claimed in claim 1, wherein the device comprises the following elements:
a number of fluid inlets (26) for the variation of a number nₛ of substances contained in a solution mixed therefrom to be administered to humans, differ from one another only in the substance quantities of the substances to be varied at the fluid outlet (8);
per fluid inlet (26), respectively at least one dosing apparatus (2,3,4,5), the latter being in fluidic connection with a common liquid line system (19);
at least one common liquid line system (19), which may comprise a liquid reservoir (1) and may contain at least one convectioning device;
at least one data input interface (7), which can receive data from the user, patient data or data from the blood treatment unit (10) and make these data available to the apparatus (9), and in particular to a control or regulating device (6);
at least one control or regulating device (6) which controls or regulates the dosing apparatuses in such a way that each base solution connected to the fluid inlet (26) is quantitatively admixed with the common liquid line system (19) so that the solution for dialysis or infusion treatments as the volume or mass flow rate required at the fluid outlet (8) and corresponds in its substance composition to the substance composition defined by the data input interface (7);
at least one fluid outlet (8) for the finally mixed solution to be administered to humans, which may likewise comprise a dosing apparatus.

3. The mixing apparatus as claimed in claim 2, which comprises at least one sensor device (18) which measures at least one physical quantity and makes this measurement result available to the control or regulating device (6).

4. The mixing apparatus (9) as claimed in claim 1, which is configured as an integral constituent of a blood treatment unit (10).

5. The mixing apparatus (9) as claimed in claim 1, which is located outside a blood treatment unit and is connected thereto.

6. The mixing apparatus (9) as claimed in claim 2, wherein the common liquid line system (19) contains a reservoir (1).

7. The mixing apparatus (9) as claimed in claim 1, wherein the number of inputs is set to four inputs for mutually independent variation of the concentration of two substances at the fluid outlet (8) of the mixing apparatus (9).

8. The mixing apparatus (9) as claimed in claim 1, wherein the number of inputs is set to two inputs for variation of the concentration of one substance at the fluid outlet (8) of the mixing apparatus (9).

9. The mixing apparatus (9) as claimed in claim 2, wherein the dosing apparatus-(26) is configured as a pump or valve.

10. The mixing apparatus (9) as claimed in claim 2, wherein the control or regulating unit (6) is configured as a programmable processor.
